(19) United States
(12) Patent Application Publication
(10) Pub. No.: US 2021/0024490 A1
(43) Pub. Date: Jan. 28, 2021

(54) METHOD FOR THE PREPARATION OF THE METABOLITES OF (4S)- AND (4R)-4-(4-CYANO-2-METHOXYPHENYL)-5-ETHOXY-2,8-DIMETHYL-1,4-DIHYDRO-1,6-NAPHTHYRIDINE-3-CARBOXAMIDE AND THE USE THEREOF

(71) Applicant: Bayer Pharma Aktiengesellschaft, Berlin (DE)

(72) Inventors: Johannes Platzek, Berlin (DE); Ludwig Zorn, Berlin (DE)

(73) Assignee: Bayer Pharma Aktiengesellschaft, Berlin (DE)

(21) Appl. No.: 17/070,371

(22) Filed: Oct. 14, 2020

Related U.S. Application Data
(63) Continuation of application No. 15/753,697, filed on Feb. 20, 2018.

(30) Foreign Application Priority Data
Aug. 21, 2015 (EP) 15182044.6

(51) Int. Cl. C07D 401/10; C07D 471/04; A61P 9/10; A61P 13/12

(52) U.S. Cl. CPC C07D 401/10; C07D 471/04; A61K 31/437; A61P 13/12; A61P 9/10

(57) ABSTRACT

The present invention relates to a novel method for preparing (4R)-4-(4-cyano-2-methoxyphenyl)-5-ethoxy-2,8-dimethyl-1,4-dihydro-1,6-naphthyridine-3-carboxamide of the formula 4R (I) and the metabolites of (4S)- and (4R)-4-(4-cyano-2-methoxyphenyl)-5-ethoxy-2,8-dimethyl-1,4-dihydro-1,6-naphthyridine-3-carboxamide of the formula (I), the formulae M1a (S), M1b (R), M2a (S), M2b (R), M3a (S) and M3b (R) and use thereof.

The present invention relates to a novel method for preparing (4R)-4-(4-cyano-2-methoxyphenyl)-5-ethoxy-2,8-dimethyl-1,4-dihydro-1,6-naphthyridine-3-carboxamide of the formula 4R (I) and the metabolites of (4S)- and (4R)-4-(4-cyano-2-methoxyphenyl)-5-ethoxy-2,8-dimethyl-1,4-dihydro-1,6-naphthyridine-3-carboxamide of the formula (I), the formulae M1a (S), M1b (R), M2a (S), M2b (R), M3a (S), M3b (R) and use thereof.

The compound of the formula 4S (I) acts as a non-steroidal antagonist of the mineralocorticoid receptor and may be used as an agent for prophylaxis and/or treatment of cardiovascular and renal disorders such as heart failure and chronic kidney disorders, for example.

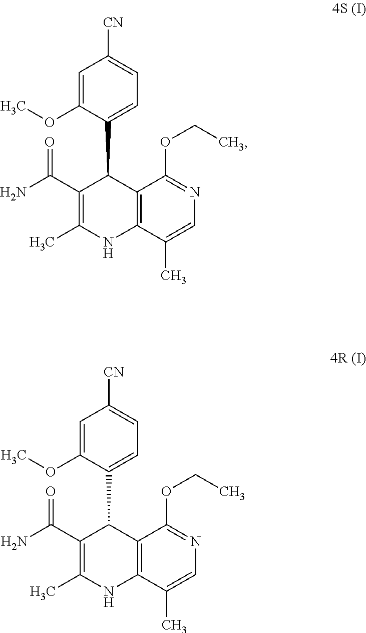 [SMILES: CCOc1ncc(C)c2c1[C@H](c1ccc(C#N)cc1OC)C(C(N)=O)=C(C)N2 ; CCOc1ncc(C)c2c1[C@@H](c1ccc(C#N)cc1OC)C(C(N)=O)=C(C)N2]

The compound of the formula 4S (I) and the preparation method thereof are described in WO2008/104306 and ChemMedChem 2012, 7, 1385, both publications disclosing a detailed discussion of the research scale synthesis.

In the publication ChemMedChem 2012, 7, 1385, which discloses the research scale synthesis of the compound of the formula (I), the compound of the formula (I) being prepared in 10 stages starting from vanillin with an overall yield of 3.76% of theory.

In the context of the clinical development of (4S)-4-(4-cyano-2-methoxyphenyl)-5-ethoxy-2,8-dimethyl-1,4-dihydro-1,6-naphthyridine-3-carboxamide (I), there existed the need for a method for preparing the main metabolites of the compound of the formula 4S (I) in order to
a) test their efficacy and
b) quantify their presence in the blood serum of the subject being tested.

For pharmacokinetic measurements, standards of very high quality had to be prepared in order to be able to carry out reliable quantification. From the structural elucidation of the metabolites (via MS of the serum of various animal species and humans) obtained after administration of (4 S)-4-(4-cyano-2-methoxyphenyl)-5-ethoxy-2,8-dimethyl-1,4-dihydro-1,6-naphthyridine-3-carboxamide of the formula 4S (I), the following 6 main metabolites were found (the absolute stereochemistry of the atropisomers can be assigned according to the Cahn-Ingold-Prelog convention and is specified in parentheses):

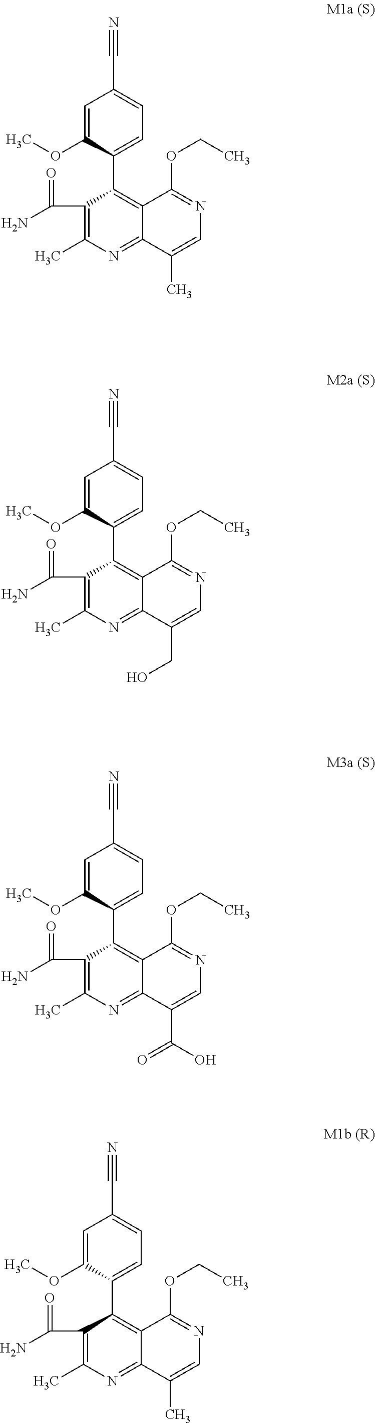

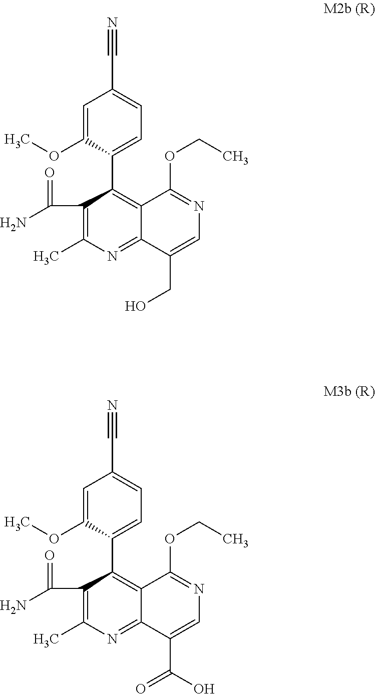

Earlier studies (A. Straub, Tetrahedron Asymmetry 12 (2001) 341-345) gave indications that the oxidized dihydropyridines, i.e. the pyridyl aryls, exhibit hindered rotation. The rotation barrier is so high that the antipodes can be separated at room temperature (axial chirality [symbol] atropisomerism). Therefore, proceeding from the racemates, preparative chiral chromatography methods were developed in order to separate these into the antipodes. This was surprisingly possible in the present case too.

Since all 6 metabolites are produced in the mammalian and human organism, there was a need for an efficient synthesis which enables the provision of relatively large amounts of the compounds of the formulae M1a (S), M2a (S), M3a (S), M1b (R), M2b (R) and M3b (R).

Starting from the racemic compound of the formula rac (I), the synthesis of which is described in the aforementioned publications,

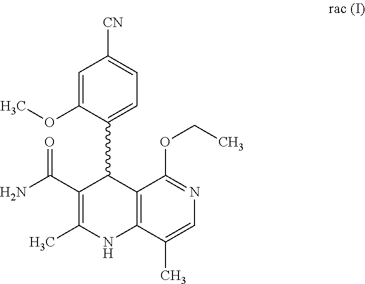 [SMILES: CCOc1ncc(C)c2c1C(c1ccc(C#N)cc1OC)C(C(N)=O)=C(C)N2]

the racemic mixture rac M1 is obtained after oxidation.

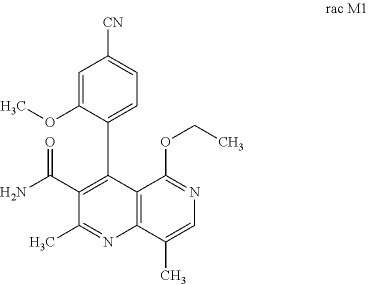 [SMILES: CCOc1ncc(C)c2nc(C)c(C(N)=O)c(-c3ccc(C#N)cc3OC)c12]

The oxidizing agents which may be used are the oxidizing agents familiar to the person skilled in the art for aromatizing piperidines and dihydropyridines, these being described, for example, in the book Pyridines: From Lab to Production; Edited by Eric F. V. Scriven, Elsevier Verlag 2013, Chapter 8, pages 116-144. Examples mentioned include DDQ in dichloromethane, chloranil in dichloromethane, manganese dioxide in dichloromethane, potassium permanganate in acetone, manganese(III) acetate in glacial acetic acid, cerium ammonium acetate in acetonitrile, pyridinium chlorochromate in dichloromethane, concentrated nitric acid in dichloromethane, iodine in methanol. Particular preference is given to DDQ or concentrated nitric acid in dichloromethane. The yields are generally very high, in general>86% of theory.

Starting from the compound rac M1, the compound rac M2 can be

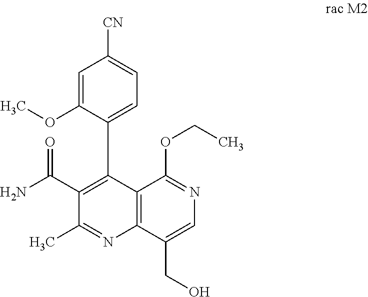 [SMILES: CCOc1ncc(CO)c2nc(C)c(C(N)=O)c(-c3ccc(C#N)cc3OC)c12]

obtained by selective hydroxylation of the methyl group. This is possible using CYP P450 expressed in E. coli, for example E. coli JM109 P450 3A4 was obtained from Oxford Biomedical Research (reactions are described in: S. P. Hanlon, T. Friedberg, C. R. Wolf, O. Ghisalba, M. Kittelmann in Modern Biooxidation: Enzymes, Reactions and Applications (Eds.: R. D. Schmid, V. B. Urlacher), Wiley-VCH, Weinheim, 2007, pp. 233-252; J. A. R. Blake, M. Pritchard, S. Ding, G. C. M. Smith, B. Burchell, C. R. Wolf, T. Friedberg, FEBS Lett. 1996, 397, 210-214; A. Parikh, E. M. J. Gillam, F. P. Guengerich, Nat. Biotechnol. 1997, 15, 784-788; Gottfried, K.; Klar, U.; Platzek, J.; Zorn, L., ChemMedChem, 2015, 10, 1240-1248; A. Parikh, E. M. J. Gillam, F. P. Guengerich, Nat. Biotechnol. 1997, 15, 784-788.). The selectivity is very high and the yields achieved (>89% of theory) are satisfactory.

Starting from the compound rac M2, the compound rac M3 can be

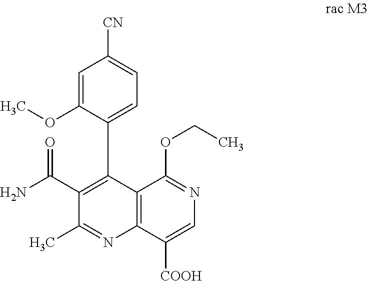 [SMILES: CCOc1ncc(C(=O)O)c2nc(C)c(C(N)=O)c(-c3ccc(C#N)cc3OC)c12]

prepared by mild oxidation of a benzylic alcohol to the acid. For this purpose, it is possible to use oxidizing agents familiar to the person skilled in the art such as Jones reagent for example. Preference is given to using Jones reagent (CrO₃ in aq. sulfuric acid). After completion of the reaction, the mixture must be quenched with, for example, isopropanol, in order to remove the excess oxidizing agent since rac M3 very readily decarboxylates to give compound (III):

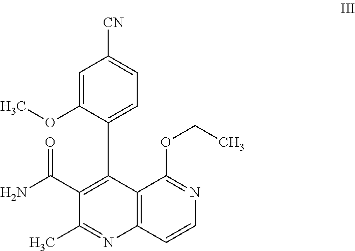 [SMILES: CCOc1nccc2nc(C)c(C(N)=O)c(-c3ccc(C#N)cc3OC)c12]

To prepare the chiral metabolites, in each case by means of chromatography on a chiral phase, the racemic mixture of rac M1 is separated into M1a and M1b, the racemic mixture of rac M2 into M2a and M2b and the racemic mixture of rac M3 into M3a and M3b. For instance, the following conditions are used for the enantiomeric separation:

| Separation | Chiral | |
|---|---|---|
| of rac Mx | stationary phase | Eluent |
| rac M1 | Chiralpak AS-H | isohexane: ethanol = |
| | (250 × 4 mm) | 50: 50 |
| rac M2 | Chiralpak AD-H | isohexane: 2-propanol = |
| | (250 × 4 mm) | 65: 35 (+0.2% |
| | | trifluoroacetic acid) |
| rac M3 | Chiralpak AD-H | isohexane: ethanol = |
| | (250 × 4 mm) | 80: 20 (+0.2% |
| | | trifluoroacetic |
| | | acid, +1% |
| | | water) |

The main fractions of the respective enantiomers are carefully concentrated (thermal stress is minimized so that no racemization occurs) and isolated.

What is surprising is the fact that the optically active compound of the formula (4S) (I) with the S configuration is metabolized mainly to M1a (S) and the subsequent metabolites M2a (S) and M3a (S) in rodents and other mammals (dog, rat, mouse), and also in humans. If the R enantiomer 4R (I) is used,

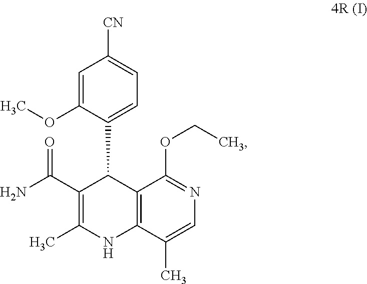 [SMILES: CCOc1ncc(C)c2c1[C@@H](c1ccc(C#N)cc1OC)C(C(N)=O)=C(C)N2]

the metabolites of the b series are principally formed, i.e. M1b (R), M2b (R) and M3b (R).

The absolute configuration was determined by means of X-ray structural analysis and by CD spectroscopy (see examples).

If, for example, an oxidation with chemical oxidizing agents is carried out, what is formed is predominantly the metabolite of the other series; compound of the formula 4S (I) (S configuration) gives rise predominantly to M1b (R); the compound of the formula 4R (I) (R configuration) gives rise predominantly to M1a (S).

In terms of their pharmacological efficacy, the metabolites are a few orders of magnitude weaker than the compound of the formula (I).

The compound of the formula (I) and metabolites thereof (M1a,b, M2a,b and M3a,b, referred to below as metabolites) act as antagonists of the mineralocorticoid receptor and exhibit an unforeseeable, valuable spectrum of pharmacological activity. They are therefore suitable for use as medicaments for treatment and/or prophylaxis of diseases in humans and animals.

The compound of the formula (I) and metabolites thereof are suitable for the prophylaxis and/or treatment of various disorders and disease-related conditions, especially of disorders characterized either by an increase in the aldosterone concentration in plasma or by a change in the aldosterone plasma concentration relative to the renin plasma concentration, or associated with these changes. Examples include: idiopathic primary hyperaldosteronism, hyperaldosteronism associated with adrenal hyperplasia, adrenal adenomas and/or adrenal carcinomas, hyperaldosteronism associated with cirrhosis of the liver, hyperaldosteronism associated with heart failure, and (relative) hyperaldosteronism associated with essential hypertension.

The compound (I) and metabolites thereof are also suitable, due to their mechanism of action, for the prophylaxis of sudden cardiac death in patients at increased risk of dying of sudden cardiac death. In particular, these are patients who suffer, for example, from any of the following disorders: primary and secondary hypertension, hypertensive heart disease with or without congestive heart failure, treatment-resistant hypertension, acute and chronic heart failure, coronary heart disease, stable and unstable angina pectoris, myocardial ischaemia, myocardial infarction, dilative cardiomyopathies, inherited primary cardiomyopathies, for example Brugada syndrome, cardiomyopathies caused by Chagas disease, shock, arteriosclerosis, atrial and ventricular arrhythmia, transient and ischaemic attacks, stroke, inflammatory cardiovascular disorders, peripheral and cardiac vascular disorders, peripheral blood flow disturbances, arterial occlusive disorders such as intermittent claudication, asymptomatic left-ventricular dysfunction, myocarditis, hypertrophic changes to the heart, pulmonary hypertension, spasms of the coronary arteries and peripheral arteries, thromboses, thromboembolic disorders, and vasculitis.

The compound (I) and metabolites thereof can also be used for the prophylaxis and/or treatment of oedema formation, for example pulmonary oedema, renal oedema or heart failure-related oedema, and of restenoses such as following thrombolysis therapies, percutaneous transluminal angioplasties (PTA) and percutaneous transluminal coronary angioplasties (PTCA), heart transplants and bypass operations.

The compound (I) and metabolites thereof are further suitable for use as a potassium-sparing diuretic and for electrolyte disturbances, for example hypercalcaemia, hypernatraemia or hypokalaemia.

The compound (I) and metabolites thereof are equally suitable for treatment of renal disorders, such as acute and chronic renal failure, hypertensive renal disease, arteriosclerotic nephritis (chronic and interstitial), nephrosclerosis, chronic renal insufficiency and cystic renal disorders, for prevention of renal damage which can be caused, for example, by immunosuppressives such as cyclosporin A in the case of organ transplants, and for renal cancer.

The compound (I) and metabolites thereof can additionally be used for the prophylaxis and/or treatment of diabetes mellitus and diabetic sequelae, for example neuropathy and nephropathy.

The compound (I) and metabolites thereof can also be used for the prophylaxis and/or treatment of microalbuminuria, for example caused by diabetes mellitus or high blood pressure, and of proteinuria.

The compound (I) and metbolites thereof are also suitable for the prophylaxis and/or treatment of disorders associated either with an increase in the plasma glucocorticoid concentration or with a local increase in the concentration of glucocorticoids in tissue (e.g. of the heart). Examples include: adrenal dysfunctions leading to overproduction of glucocorticoids (Cushing's syndrome), adrenocortical tumours with resulting overproduction of glucocorticoids, and pituitary tumours which autonomously produce ACTH (adrenocorticotropic hormone) and thus lead to adrenal hyperplasias with resulting Cushing's disease.

The compound (I) and metabolites thereof can additionally be used for the prophylaxis and/or treatment of obesity, of metabolic syndrome and of obstructive sleep apnoea.

The compound (I) and metabolites thereof can also be used for the prophylaxis and/or treatment of inflammatory disorders caused for example by viruses, spirochetes, fungi, bacteria or mycobacteria, and of inflammatory disorders of unknown etiology, such as polyarthritis, lupus erythematosus, peri- or polyarteritis, dermatomyositis, scleroderma and sarcoidosis.

The compound (I) and metabolites thereof can further be used for the treatment of central nervous disorders such as depression, states of anxiety and chronic pain, especially migraine, and for neurodegenerative disorders such as Alzheimer's disease and Parkinson's syndrome.

The compound (I) and metabolites thereof are also suitable for the prophylaxis and/or treatment of vascular damage, for example following procedures such as percutaneous transluminal coronary angioplasty (PTCA), implantation of stents, coronary angioscopy, reocclusion or restenosis following bypass operations, and for endothelial dysfunction, for Raynaud's disease, for thromboangiitis obliterans (Buerger's syndrome) and for tinnitus syndrome.

The present invention further relates to the use of the compound (I) and metabolites thereof for treatment and/or prevention of disorders, especially of the aforementioned disorders.

The present invention further relates to the use of the compound (I) and metabolites thereof for preparing a medicament for treatment and/or prevention of disorders, especially of the aforementioned disorders.

Further subject matter is a method for treatment and/or prevention of disorders, especially of the aforementioned disorders, using an effective amount of at least one of the compounds according to the invention.

The compound (I) may be used alone or, if required, in combination with other active ingredients. Further subject matter are medicaments, comprising a compound (I) and/or one or more metabolites and one or more further active ingredients, especially for the treatment and/or prevention of the aforementioned disorders. Preferred examples of suitable combination active ingredients include:
active ingredients which lower blood pressure, for example and with preference from the group of calcium antagonists, angiotensin AII antagonists, ACE inhibitors, endothelin antagonists, renin inhibitors, alpha-receptor blockers, beta-receptor blockers and Rho kinase inhibitors;
diuretics, especially loop diuretics, and thiazides and thiazide-like diuretics;
antithrombotic agents, by way of example and with preference from the group of the platelet aggregation inhibitors, the anticoagulants or the profibrinolytic substances;
active ingredients which alter lipid metabolism, for example and with preference from the group of thyroid receptor agonists, cholesterol synthesis inhibitors, preferred examples being HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists;
organic nitrates and NO donors, for example sodium nitroprusside, nitroglycerin, isosorbide mononitrate, isosorbide dinitrate, molsidomine or SIN-1, and inhaled NO;
compounds having a positive inotropic effect, for example cardiac glycosides (digoxin), beta-adrenergic and dopaminergic agonists such as isoproterenol, adrenaline, noradrenaline, dopamine and dobutamine;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, and PDE 3 inhibitors such as amrinone and milrinone;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
calcium sensitizers, a preferred example being levosimendan;
NO-independent but haem-dependent stimulators of guanylate cyclase, such as especially the compounds described in WO 00/06568, WO 00/06569, WO 02/42301 and WO 03/095451,
NO— and haem-independent activators of guanylate cyclase, such as especially the compounds described in WO 01/19355, WO 01/19776, WO 01/19778, WO 01/19780, WO 02/070462 and WO 02/070510;
inhibitors of human neutrophil elastase (HNE), for example sivelestat or DX-890 (Reltran);
compounds which inhibit the signal transduction cascade, for example tyrosine kinase inhibitors, especially sorafenib, imatinib, gefitinib and erlotinib; and/or
compounds which influence the energy metabolism of the heart, preferred examples being etomoxir, dichloroacetate, ranolazine or trimetazidine.

In a preferred embodiment, the compound (I) and metabolites thereof are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Agents which lower blood pressure are preferably understood to mean compounds from the group of calcium antagonists, angiotensin AII antagonists, ACE inhibitors, endothelin antagonists, renin inhibitors, alpha-receptor blockers, beta-receptor blockers, Rho kinase inhibitors, and the diuretics.

In a preferred embodiment of the invention, the compound (I) and/or one or more metabolites thereof is administered in combination with a calcium antagonist, by way of example and with preference nifedipine, amlodipine, verapamil or diltiazem.

In a preferred embodiment, the compound (I) and/or one or more metabolites thereof is administered in combination with an angiotensin AII antagonist, by way of example and with preference losartan, candesartan, valsartan, telmisartan or embusartan.

In a preferred embodiment, the compound (I) and/or one or more metabolites thereof is administered in combination with an ACE inhibitor, by way of example and with preference enalapril, captopril, lisinopril, ramipril, delapril, fosinopril, quinopril, perindopril or trandopril.

In a preferred embodiment, the compound (I) and/or one or more metabolites thereof is administered in combination with an endothelin antagonist, by way of example and with preference bosentan, darusentan, ambrisentan or sitaxsentan.

In a preferred embodiment, the compound (I) and/or one or more metabolites thereof is administered in combination with a renin inhibitor, by way of example and with preference aliskiren, SPP-600, SPP-635, SPP-676, SPP-800 or SPP-1148.

In a preferred embodiment, the compound (I) and/or one or more metabolites thereof is administered in combination with an alpha-1 receptor blocker, by way of example and with preference prazosin.

In a preferred embodiment, the compound (I) and/or one or more metabolites thereof is administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment, the compound (I) and/or one or more metabolites thereof is administered in combination with a Rho kinase inhibitor, by way of example and with preference fasudil, Y-27632, SLx-2119, BF-66851, BF-66852, BF-66853, KI-23095 or BA-1049.

Agents having antithrombotic activity (antithrombotics) are understood to mean compound (I) and/or one or more metabolites thereof, preferably from the group of thrombocyte aggregation inhibitors, anticoagulants and profibrinolytic substances.

In a preferred embodiment, the compound (I) and/or one or more metabolites thereof is administered in combination with a thrombocyte aggregation inhibitor, by way of example and with preference aspirin, clopidogrel, ticlopidine or dipyridamole.

In a preferred embodiment, the compound (I) and/or one or more metabolites thereof is administered in combination with a thrombin inhibitor, by way of example and with preference ximelagatran, melagatran, bivalirudin or clexane.

In a preferred embodiment of the invention, the compound (I) is administered in combination with a GPIIb/IIIa antagonist, by way of example and with preference tirofiban or abciximab.

In a preferred embodiment, the compound (I) and/or one or more metabolites thereof is administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment, the compound (I) and/or one or more metabolites thereof is administered in combination with heparin or with a low molecular weight (LMW) heparin derivative.

In a preferred embodiment, the compound (I) and/or one or more metabolites thereof is administered in combination with a vitamin K antagonist, by way of example and with preference coumarin.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists.

In a preferred embodiment, the compound (I) and/or one or more metabolites thereof is administered in combination with a CETP inhibitor, by way of example and with preference torcetrapib (CP-529 414), JJT-705, BAY 60-5521, BAY 78-7499 or CETP vaccine (Avant).

In a preferred embodiment, the compound (I) and/or one or more metabolites thereof is administered in combination with a thyroid receptor agonist, by way of example and with preference D-thyroxine, 3,5,3′-triiodothyronine (T3), CGS 23425 or axitirome (CGS 26214).

In a preferred embodiment, the compound (I) and/or one or more metabolites thereof is administered in combination with an HMG-CoA reductase inhibitor from the class of statins, by way of example and with preference lovastatin, simvastatin, pravastatin, fluvastatin, atorvastatin, rosuvastatin, cerivastatin or pitavastatin.

In a preferred embodiment, the compound (I) and/or one or more metabolites thereof is administered in combination with a squalene synthesis inhibitor, by way of example and with preference BMS-188494 or TAK-475.

In a preferred embodiment, the compound (I) and/or one or more metabolites thereof is administered in combination with an ACAT inhibitor, by way of example and with preference avasimibe, melinamide, pactimibe, eflucimibe or SMP-797.

In a preferred embodiment, the compound (I) and/or one or more metabolites thereof is administered in combination with an MTP inhibitor, by way of example and with preference implitapide, BMS-201038, R-103757 or JTT-130.

In a preferred embodiment of the invention, the compound (I) and/or one or more metabolites thereof is administered in combination with a PPAR-gamma agonist, by way of example and with preference pioglitazone or rosiglitazone.

In a preferred embodiment, the compound (I) and/or one or more metabolites thereof is administered in combination with a PPAR-delta agonist, by way of example and with preference GW-501516 or BAY 68-5042.

In a preferred embodiment, the compound (I) and/or one or more metabolites thereof is administered in combination with a cholesterol absorption inhibitor, by way of example and with preference ezetimibe, tiqueside or pamaqueside.

In a preferred embodiment, the compound (I) and/or one or more metabolites thereof is administered in combination with a lipase inhibitor, by way of example and with preference orlistat.

In a preferred embodiment, the compound (I) and/or one or more metabolites thereof is administered in combination with a bile acid adsorber, by way of example and with preference cholestyramine, colestipol, colesolvam, cholestagel or colestimide.

In a preferred embodiment, the compound (I) and/or one or more metabolites thereof is administered in combination with a bile acid reabsorption inhibitor, by way of example and with preference ASBT (=IBAT) inhibitors such as, e.g. AZD-7806, S-8921, AK-105, BARI-1741, SC-435 or SC-635.

In a preferred embodiment, the compound (I) and/or one or more metabolites thereof is administered in combination with a lipoprotein(a) antagonist, by way of example and with preference gemcabene calcium (CI-1027) or nicotinic acid.

Further subject matter are medicaments which comprise a compound of the formula (I) and/or one or more metabolites thereof, typically together with one or more inert, non-toxic, pharmaceutically suitable excipients, and the use thereof for the aforementioned purposes.

The compound (I) and metabolites thereof can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compound (I) and metabolites thereof can be administered in administration forms suitable for these administration routes.

Suitable administration forms for oral administration are those which work according to the prior art and release the compound (I) and metabolites thereof rapidly and/or in a modified manner and which contain the compound according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include inter alia preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Oral and parenteral administration are preferred, especially oral and intravenous administration.

The compound (I) and metabolites thereof can be converted to the administration forms mentioned. This can be accomplished in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctors.

In general, it has been found to be advantageous in the case of parenteral administration to administer amounts of about 0.001 to 1 mg/kg, preferably about 0.01 to 0.5 mg/kg body weight to achieve effective results. In the case of oral administration the dosage is about 0.01 to 100 mg/kg, preferably about 0.01 to 20 mg/kg and most preferably 0.1 to 10 mg/kg body weight.

It may nevertheless be necessary in some cases to deviate from the stated amounts, and specifically as a function of body weight, route of administration, individual response to the active ingredient, nature of the preparation and time at which or interval over which administration takes place. Thus in some cases it may be sufficient to manage with less than the aforementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for liquid/liquid solutions are based in each case on volume.

The present invention therefore relates to compounds of the formulae

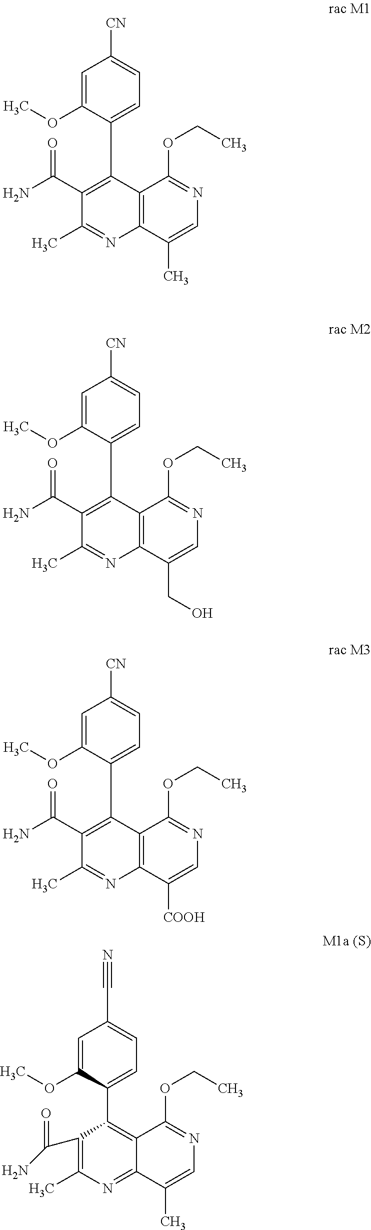

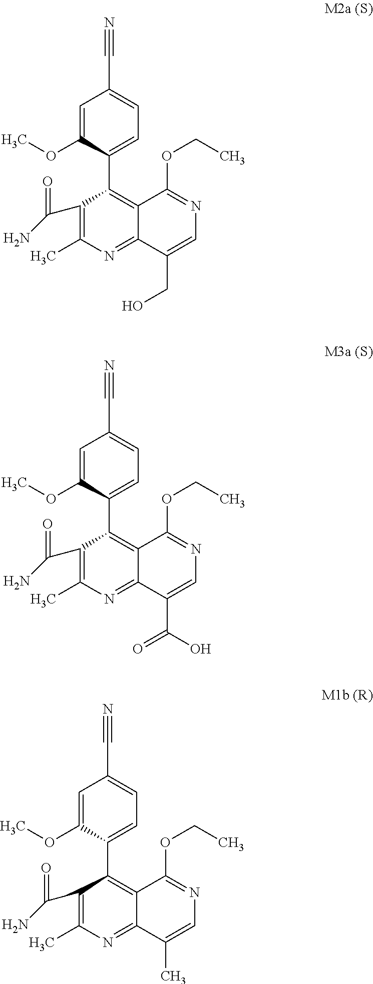

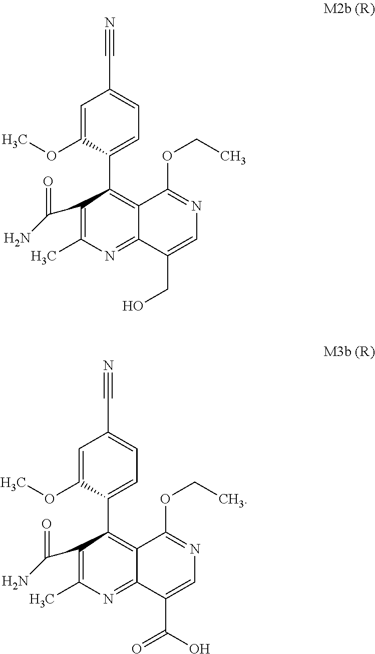

The present invention further relates to a method for preparing the compound of the formulae M1a (S) and M1b (R)

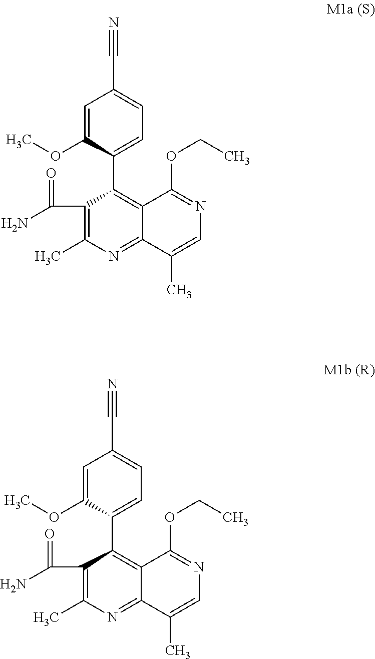

characterized in that the compound of the formula rac M1

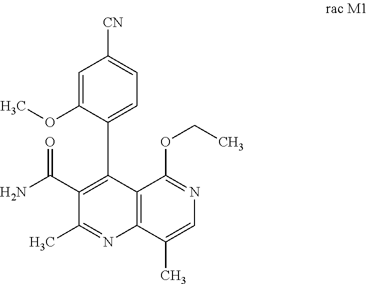 [SMILES: CCOc1ncc(C)c2nc(C)c(C(N)=O)c(-c3ccc(C#N)cc3OC)c12]

is prepared by oxidation of the compound of the formula rac (I)

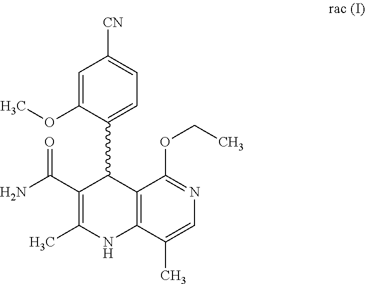 [SMILES: CCOc1ncc(C)c2c1C(c1ccc(C#N)cc1OC)C(C(N)=O)=C(C)N2]

and the racemate is separated into the enantiomers of the formulae M1a (S) and M1b (R) by chromatographic methods on a chiral phase.

The present invention further relates to a method for preparing the compound of the formulae M2a (S) and M2b (R)

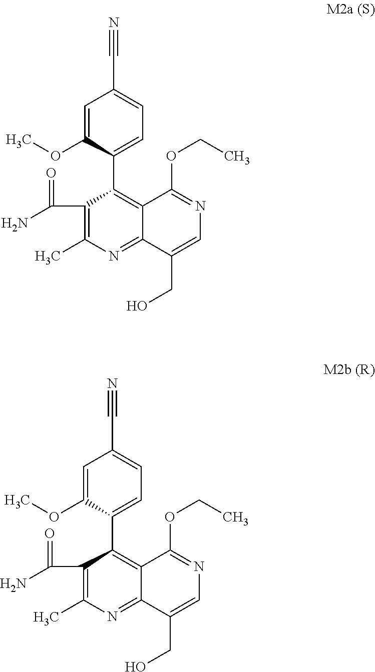

characterized in that the compound of the formula rac M2

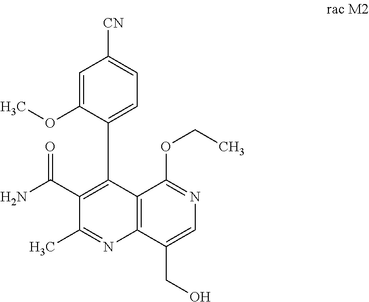 [SMILES: CCOc1ncc(CO)c2nc(C)c(C(N)=O)c(-c3ccc(C#N)cc3OC)c12]

is prepared by selective hydroxylation of the methyl group of the compound of the formula rac M1

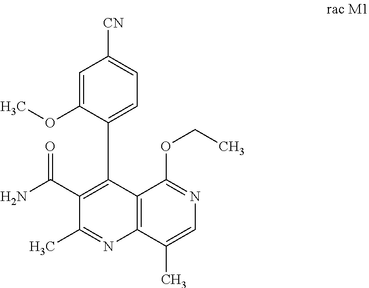 [SMILES: CCOc1ncc(C)c2nc(C)c(C(N)=O)c(-c3ccc(C#N)cc3OC)c12]

and the racemate is separated into the enantiomers of the formulae M2a (S) and M2b (R) by chromatographic methods on a chiral phase.

The present invention further relates to a method for preparing the compound of the formulae M3a (S) and M3b (R)

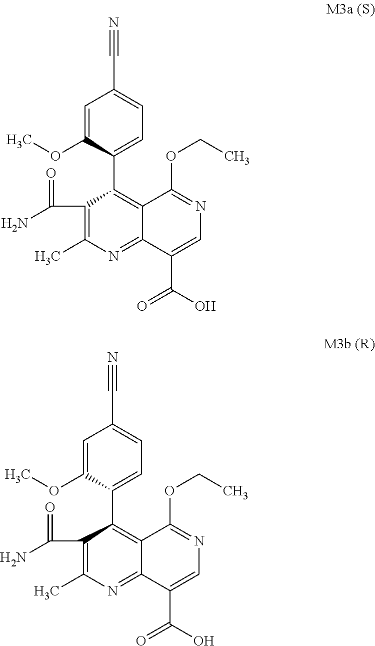

characterized in that the compound of the formula rac M3

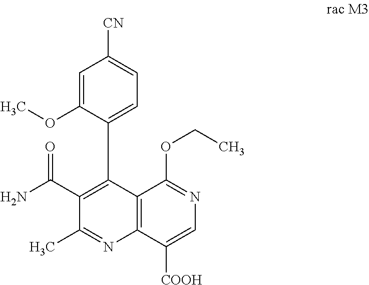 [SMILES: CCOc1ncc(C(=O)O)c2nc(C)c(C(N)=O)c(-c3ccc(C#N)cc3OC)c12]

is prepared by oxidation of the benzylic alcohol of the compound of the formula rac M2

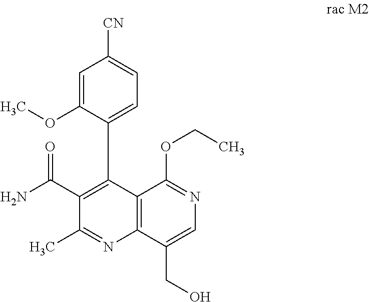 [SMILES: CCOc1ncc(CO)c2nc(C)c(C(N)=O)c(-c3ccc(C#N)cc3OC)c12]

and the racemate is separated into the enantiomers of the formulae Mia (S) and M3b (R) by chromatographic methods on a chiral phase.

### Experimental Section Abbreviations and Acronyms

MS: mass from mass spectrometry
HPLC: high-performance liquid chromatography

### EXAMPLES

### Example 1

### Preparation of the Compound of the Formula Rac M1

### Rac 4-(4-cyano-2-methoxyphenyl)-5-ethoxy-2,8-dimethyl-1,6-naphthyridine-3-carboxamide

100.00 g (264.25 mmol) of 4(R,S)-4-(4-cyano-2-methoxyphenyl)-5-ethoxy-2,8-dimethyl-1,4-dihydro-1,6-naphthyridine-3-carboxamide (rac I) were initially charged in 4 kg of dichloromethane, and 68.98 g (303.88 mmol) of 2,3-dichloro-5,6-dicyano-1,4-benzoquinone (DDQ) were added at 20° C. The mixture was stirred at 20° C. for 1 h. The precipitated solid was filtered off and washed twice with 400 g each time of dichloromethane. The mixture was concentrated to dryness under reduced pressure and the residue was taken up in 1200 g of ethanol. The mixture was heated to reflux and about 800 g of ethanol were distilled off. The mixture was left to cool down to room temperature and stirred at 20° C. for a further 1 h. The product was filtered off, washed with a little ethanol (about 80 g), and dried under reduced pressure overnight (50° C.).

Yield: 85.80 g (86.04% of theory) of a beige solid.

MS (EIpos): m/z=378 [M+H]⁺

¹H NMR (500 MHz, DMSO-d6): δ=0.72 (t, 3H), 2.50 (s, 3H), 2.70 (s, 3H), 3.65 (s, 1H), 4.00 (m (broad), 2H), 7.30 (d, 1H), 7.45 (d, 1H), 7.50 (s, 2H), 7.69 (s, 1H), 8.05 (s, 1H)

### Enantiomer Separation on a Chiral Phase

2.00 g of the compound of the formula rac-M1were separated on a chiral phase

Chiral phase: Chiralpak AS-H (250×4 mm)

Eluent: isohexane:ethanol=50:50

Yield of the compound of the formula M1a (S): 0.91 g of (S)-4-(4-cyano-2-methoxyphenyl)-5-ethoxy-2,8-dimethyl-1,6-naphthyridine-3-carboxamide

HPLC-Method: RT ca. 6.08 min.

MS (EIpos): m/z=378 [M+H]+

¹H NMR (500 MHz, DMSO-d6): δ=0.72 (t, 3H), 2.50 (s, 3H), 2.70 (s, 3H), 3.65 (s, 1H), 4.00 (m (broad), 2H), 7.30 (d, 1H), 7.45 (d, 1H), 7.50 (s, 2H), 7.69 (s, 1H), 8.05 (s, 1H)

Yield of the compound of the formula M1b (R): 0.90 g of (R)-4-(4-cyano-2-methoxyphenyl)-5-ethoxy-2,8-dimethyl-1,6-naphthyridine-3-carboxamide

HPLC-Method: RT ca. 9.03 min.

MS (EIpos): m/z=378 [M+H]+

¹H NMR (500 MHz, DMSO-d6): δ=0.72 (t, 3H), 2.50 (s, 3H), 2.70 (s, 3H), 3.65 (s, 1H), 4.00 (m (broad), 2H), 7.30 (d, 1H), 7.45 (d, 1H), 7.50 (s, 2H), 7.69 (s, 1H), 8.05 (s, 1H)

### Example 2

### Preparation of the Compound of the Formula Rac M2

### Rac 4-(4-cyano-2-methoxyphenyl)-5-ethoxy-8-(hydroxymethyl)-2-methyl-1,6-naphthyridine-3-carboxamide

E. coli JM109 P450 3A4 was obtained from Oxford Biomedical Research.

Oxford trace element solution for 1 l final volume: iron trichloride hexahydrate (27 g l⁻¹), zinc dichloride (1.31 g l⁻¹), cobalt dichloride hexahydrate (2.87 g l⁻¹), copper dichloride dihydrate (1.27 g l⁻¹), boric acid (0.5 g l⁻¹), calcium dichloride dihydrate (1.32 g l⁻¹), disodium molybdate dihydrate (2.35 g l⁻¹) and 37% hydrochloric acid (100 ml) in water.

Two 500 ml Erlenmeyer flasks were sterilized with a nutrient solution (each 100 ml) in an autoclave at 121° C. for 20 minutes. The nutrient solution consisted of tryptone (16 g l⁻¹), sodium chloride (10 g l⁻¹) and yeast extract (10 g l⁻¹) and was adjusted to a pH of 7.2-7.4 with 16% sodium hydroxide solution. After the sterilization process, ampicillin (100 mg l⁻¹) was added to the cooled flasks. Both 500 ml Erlenmeyer flasks were each inoculated with a glycerol cryoculture (50 μl) of the E. coli strain JM 109 P450 3A4. The flasks were shaken at 37° C. and 165 rpm for 17 hours.

A 20 l fermenter was charged with tryptone (12 g l⁻¹), yeast extract (24 g l⁻¹), peptone from meat (2 g l⁻¹) [tryptic digest], potassium dihydrogen phosphate (2.2 g l⁻¹), dipotassium hydrogen phosphate (9.4 g l⁻¹), and 87% glycerol (4.6 g l⁻¹). The medium was sterilized in the fermenter at 121° C. for 40 minutes. The following solutions were added at 37° C.: ampicillin (2.0 g) in water (20 ml), riboflavin (20 mg) in water (20 ml), thiamine hydrochloride (6.74 g) in water (10 ml) and Oxford trace element solution (5 ml). After 2 hours, the fermenter was inoculated with the pre-culture from the two 500 ml Erlenmeyer flasks. The fermenter was stirred at 250 rpm and 6.6 l min⁻¹ air at pH 6.6. The pH was regulated using 16% sodium hydroxide solution and 16% phosphoric acid. After 2 hours and 15 minutes, the temperature was lowered to 25° C. since the optical density (OD550) of 0.89 had been attained. 10 minutes later, IPTG (4.76 g, isopropyl beta-D-thiogalactopyranoside) in water (40 ml) and 5-aminolevulinic acid (1.676 g) in water (40 ml) were added. After a further 6 hours and 35 minutes, the pH decreased and the phosphoric acid solution was replaced by an aqueous glucose solution (50% glucose, sterile filtered). The aqueous glucose solution was then metered in in order to maintain the pH at 6.6. After 120 hours, the cell culture was harvested by centrifuge. The harvested cells (1312.5 g) were resuspended in cryobuffer (cryobuffer: dipotassium hydrogen phosphate (12.3 g l⁻¹), potassium dihydrogen phosphate (4 g l⁻¹), glucose (100 ml l⁻¹, 50% aqueous solution), 0.5M EDTA, glycerol (40 ml l⁻¹, 87%, 1313 ml) and stored at −80° C.

A 100 l fermenter was charged with water (94 l), dipotassium hydrogen phosphate (1.23 kg), potassium dihydrogen phosphate (400 g) and Synperonic (2.5 ml). The amount of buffer salts in this case was calculated at 0.1M at a volume of 100 l. Subsequently, the fermenter was sterilized at 121° C. for 40 minutes. The volume after sterilization was 97 l. An aqueous glucose solution (2 l, 50% glucose, sterile filtered) and an EDTA solution (100 ml of a 0.5M solution; final concentration 0.5 mM at a volume of 100 l) were added. Subsequently, the reactant (5 g, 13.28 mmol) was dissolved in DMF (200 ml) and added to the fermenter. The fermenter was stirred at 70 rpm and 33.3 l min⁻¹ air. The pH was maintained at 7.4 by addition of 16% aqueous sodium hydroxide solution. At intervals of 15 minutes each, cryopreserved cells (in each case 1200 ml in 50% glycerol) were added three times. The oxygen partial pressure was maintained at 50% by the stirring speed. After 3 hours, the culture broth was harvested.

The culture broth was stirred with methyl isobutyl ketone (50 l) for 18 hours. The phases were separated and the aqueous phase was again stirred (32 rpm) with methyl isobutyl ketone (15 l) for 19 hours. The organic phases were concentrated separately. The concentrates were combined and concentrated to dryness. The solid residue was heated to reflux in methanol (200 ml). The mixture was cooled and stored overnight in a refrigerator. The residue was filtered off under suction, washed with a little methanol and dried under reduced pressure at room temperature.

Yield: 4.79 g (89% of theory) of a beige-white solid.

MS (EIpos): m/z=393 [M+H]⁺

¹H NMR (400 MHz, DMSO-d6) δ ppm=0.71 (t, 3H) 2.68 (s, 3H) 3.65 (s, 3H) 3.91-4.01 (m, 1H) 4.01-4.10 (m, 1H) 4.96 (m, 2H) 5.02-5.14 (s-br, 1H) 7.31 (d, 1H) 7.44 (dd, 1H) 7.47-7.52 (m, 2H) 7.70 (s, 1H) 8.15 (s, 1H).

### Enantiomer Separation on a Chiral Phase

2.00 g of the compound of the formula rac-M2 were separated on a chiral phase:

Chiral phase: Chiralpak AD-H (250×4 mm)

Eluent: isohexane:2-propanol=65:35 (+0.2% trifluoroacetic acid)

Yield of the compound of the formula M2a (S): 0.87 g of (S)-4-(4-cyano-2-methoxyphenyl)-5-ethoxy-8-(hydroxymethyl)-2-methyl-1,6-naphthyridine-3-carboxamide

HPLC-Method: RT ca. 4.33 min.

MS (EIpos): m/z=393 [M+H]+

¹H NMR (400 MHz, DMSO-d6) δ ppm=0.71 (t, 3H) 2.68 (s, 3H) 3.65 (s, 3H) 3.91-4.01 (m, 1H) 4.01-4.10 (m, 1H) 4.96 (m, 2H) 5.02-5.14 (s-br, 1H) 7.31 (d, 1H) 7.44 (dd, 1H) 7.47-7.52 (m, 2H) 7.70 (s, 1H) 8.15 (s, 1H).

Yield of the compound of the formula M2b (R): 0.85 g of (R)-4-(4-cyano-2-methoxyphenyl)-5-ethoxy-8-(hydroxymethyl)-2-methyl-1,6-naphthyridine-3-carboxamide

HPLC-Method: RT ca. 6.55 min.

MS (EIpos): m/z=393 [M+H]+

¹H NMR (400 MHz, DMSO-d6) δ ppm=0.71 (t, 3H) 2.68 (s, 3H) 3.65 (s, 3H) 3.91-4.01 (m, 1H) 4.01-4.10 (m, 1H) 4.96 (m, 2H) 5.02-5.14 (s-br, 1H) 7.31 (d, 1H) 7.44 (dd, 1H) 7.47-7.52 (m, 2H) 7.70 (s, 1H) 8.15 (s, 1H).

### Example 3

### Preparation of the Compound of the Formula Rac M3

### Rac 3-carbamoyl-4-(4-cyano-2-methoxyphenyl)-5-ethoxy-2-methyl-1,6-naphthyridine-8-carboxylic Acid

2.50 g (6.371 mmol) of the compound of the formula M2 were suspended in 75 ml of acetone, and the mixture was cooled to 0° C. 5 ml of Jones reagent were added (prepared from 2.30 g of chromium(VI) trioxide in 2.3 ml of conc. sulfuric acid and dissolved in 5 ml of water). The reaction was monitored by HPLC (see below). As soon as the starting material was <1%, 25 ml of isopropanol were added and the mixture was stirred overnight. 500 ml of dichloromethane and 100 ml of methanol were added and the greenish precipitate (chromium salts!) was filtered off. The filtrate was concentrated to dryness under reduced pressure.

Yield: 2.20 g (84.94% of theory) of a yellowish solid.

HPLC-Method A: RT ca. 5.1 min.

HPLC conditions/method

Method A

YMC Hydrosphere C18

150*4.6 mm, 3.0 μm

25° C., 1 ml/min, 270 nm, 4 nm

0′: 70% TFA 0.1%*; 30% acetonitrile

17′: 20% TFA 0.1%*; 80% acetonitrile

18′: 70% TFA 0.1%*; 30% acetonitrile

*: TFA in water

MS (EIpos): m/z=407 [M+H]+

¹H-NMR (500 MHz, DMSO-d6): δ=0.75 (t, 3H), 2.80 (s, 3H), 3.67 (s, 3H), 4.17 (m(broad), 2H), 7.36 (d, 1H), 7.50 (d, 1H), 7.60 (s, 1H), 7.71 (s, 1H), 7.85 (s, 1H), 8.95 (s, 1H), 15.40 (s(broad), 1H)

### Enantiomer Separation on a Chiral Phase

2.00 g of the compound of the formula rac-M3 were separated on a chiral phase

Chiral phase: Chiralpak AD-H (250×4 mm)

Eluent: isohexane:ethanol=80:20 (+0.2% trifluoroacetic acid, +1% water)

Yield M3a: 0.85 g of (S)-3-carbamoyl-4-(4-cyano-2-methoxyphenyl)-5-ethoxy-2-methyl-1,6-naphthyridine-8-carboxylic acid

HPLC-Method: RT ca. 6.97 min.

MS (EIpos): m/z=407 [M+H]+

¹H-NMR (500 MHz, DMSO-d6): δ=0.75 (t, 3H), 2.80 (s, 3H), 3.67 (s, 3H), 4.17 (m(broad), 2H), 7.36 (d, 1H), 7.50 (d, 1H), 7.60 (s, 1H), 7.71 (s, 1H), 7.85 (s, 1H), 8.95 (s, 1H), 15.40 (s(broad), 1H)

Yield of the compound of the formula M3b (R): 0.83 g of (R)-3-carbamoyl-4-(4-cyano-2-methoxyphenyl)-5-ethoxy-2-methyl-1,6-naphthyridine-8-carboxylic acid

HPLC-Method: RT ca. 8.63 min.

MS (EIpos): m/z=407 [M+H]+

¹H-NMR (500 MHz, DMSO-d6): δ=0.75 (t, 3H), 2.80 (s, 3H), 3.67 (s, 3H), 4.17 (m(broad), 2H), 7.36 (d, 1H), 7.50 (d, 1H), 7.60 (s, 1H), 7.71 (s, 1H), 7.85 (s, 1H), 8.95 (s, 1H), 15.40 (s(broad), 1H)

### Example 4

### Single-Crystal x-Ray Structure Analysis of the Compound of the Formula M1b (R): (R)-4-(4-cyano-2-methoxyphenyl)-5-ethoxy-2,8-dimethyl-1,6-naphthyridine-3-carboxamide

Analysis method: Single-crystal x-ray structure analysis

Crystal analysed: colourless block, 0.40×0.20×0.20 mm³

Experimental:

The crystal structure determination was conducted with the aid of a diffractometer (Oxford Diffraction, Xcalibur series), equipped with a CCD area detector (Ruby model), a sealed x-ray tube with CuKa radiation, osmium reflector as monochromator and a cryojet cooling device for low-temperature measurements (T=100 K).

360° data collection, omega and phi scan. Programs used: Data recording and reduction with Crysalis (Oxford Diffraction 2007). The crystal structure solution was conducted by means of direct methods as implemented in SHELXTL Version 6.10 (Sheldrick, University of Gottingen (Germany), 2000), and visualized by means of the XP program. Missing atoms were subsequently localized with the aid of difference Fourier synthesis and added to the atom list. The refinement by the method of least mean squares to F2 was conducted with all intensities measured and conducted with the program SHELXTL Version 6.10 (Sheldrick, University of Gottingen (Germany), 2000). All non-hydrogen atoms were refined, including anisotropic deflection parameters.

Crystal data and structure refining of the compound of the formula M1b (R): (R)-4-(4-cyano-2-methoxyphenyl)-5-ethoxy-2,8-dimethyl-1,6-naphthyridine-3-carboxamide

Identification code: M1b

Empirical formula: C21 H20 N4 O3

Molecular mass: 376.41

Temperature: 100 K

Wavelength: 1.54178 Å

Crystal system: orthorhombic

Space group: P2(1)2(1)2(1)

Lattice constants: a=9.70950(10) Ålattice

b=10.67390(10) Å?=90°.

c=18.9480(2) Å?=90°.

Volume: 1963.74(3) Å₃

Z4

Specific density (calculated): 1.273 Mg/m₃

Absorption coefficient: 0.714 mm₋₁

F(000) 792

Crystal dimensions: 0.40×0.20×0.20 mm₃

Theta range for data recording: 4.67 to 65.66°.

Index range: −11≤h≤9, −12≤k≤12, −19≤I≤22

Reflections recorded: 15493

Independent reflections: 3367 [R(int)=0.0230]

Completeness at theta=65.66° 99.5%

Absorption correction: crysalis

Refinement method: full matrix method of least mean squares to F₂

Data/restrictions/parameters: 3367/0/257

Quality of fit to F₂: 1.048

Final R values: [I>2sigma(I)] R1=0.0242, wR2=0.0636

R values (all data): R1=0.0249, wR2=0.0641

Absolute structure parameter: −0.18(13)

Greatest and smallest differential density: 0.142 and −0.139 e.Å₋₃

### X-Ray Structure Analysis:

The x-ray structure analysis showed that, when the 1,6-naphthyridine-3-carboxamide ring system is in the plane of the paper, the 4-cyano-2-methoxyphenyl substituent is at right angles thereto, in which case the methoxy group is then behind the plane of the paper.

### Determination of Absolute Configuration

| Chirality test* | Correct structure | | Inverted structure | |
|---|---|---|---|---|
| Flack parameter | −0.1838 | (0.1347) | 1.1745 | (0.1364) |
| (standard deviation) | | | | |
| Twin Basf (standard | 0.0000 | (0.1348) | 1.1855 | (0.1347) |
| deviation) | | | | |

| | | |
|---|---|---|
| wR2 value (with | 0.0641 | 0.0649 |
| Flack parameter) | | |
| Chirality | Ra | Sa |

H. D. Flack, Acta Cryst., 1983, A39, 876-881
H. D. Flack, G. Bernardinelli, Acta Cryst., 1999, A55, 908-915
H. D. Flack, G. Bernardinelli, J. Appl. Cryst., 2000, 33, 1143-1148.

The compound of the formula M1b (R) thus has the absolute configuration R (Ra).

The naming of the absolute configuration follows the Cahn-Ingold-Prelog rules for compounds having axial chirality.

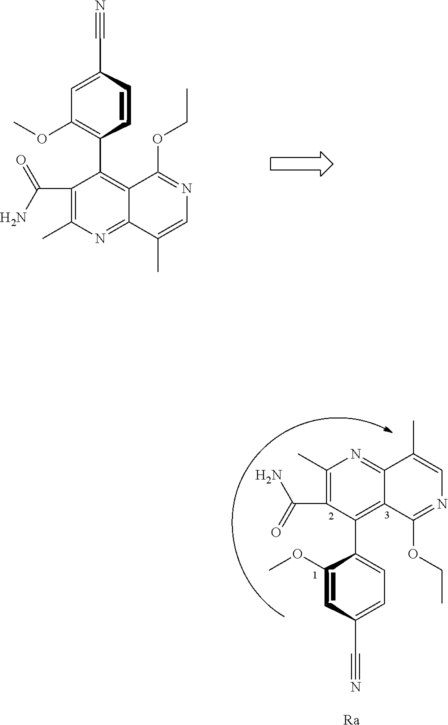

### Example 5

### Determination of the Absolute Configuration of the Mb (R) Series by Correlation of the CD Spectra

FIGS. 3-5 show the CD spectra of the compounds of the formulae M1b (R), M2b (R) and M3b (R).

Conclusion: Owing to the identical pattern sequence of the Cotton effects, the metabolites of the Mb (R) series have the same absolute configuration. The reverse applies equally to the Ma (S) series.

B-1. Cellular In Vitro Assay to Determine the Inhibitory MR Activity and MR Selectivity Compared with Other Steroid Hormone Receptors

Antagonists of the human mineralocorticoid receptor (MR) are identified, and the efficacy of the compounds described herein is quantified with the aid of a recombinant cell line. The cell is originally derived from a hamster ovary epithelial cell (Chinese Hamster Ovary, CHO K1, ATCC: American Type Culture Collection, VA 20108, USA).

An established chimera system in which the ligand-binding domains of human steroid hormone receptors are fused to the DNA-binding domain of the yeast transcription factor GAL4 is used in this CHO K1 cell line. The GAL4-steroid hormone receptor chimeras produced in this way are cotransfected and stably expressed with a reporter construct in the CHO cells.

### Cloning:

To generate the GAL4-steroid hormone receptor chimeras, the GAL4 DNA-binding domain (amino acids 1-147) from the vector pFC2dbd (from Stratagene) is cloned with the PCR-amplified ligand-binding domains of the mineralocorticoid receptor (MR, amino acids 734-985), of the glucocorticoid receptor (GR, amino acids 443-777), of the progesterone receptor (PR, amino acids 680-933) and of the androgen receptor (AR, amino acids 667-919) into the vector pIRES2 (from Clontech). The reporter construct, which contains five copies of the GAL4 binding site upstream of a thymidine kinase promoter, leads to expression of firefly luciferase (l'hotinus pyralis) after activation and binding of the GAL4-steroid hormone receptor chimeras by the respective specific agonists aldosterone (MR), dexamethasone (GR), progesterone (PR) and dihydrotestosterone (AR).

### Assay Procedure:

The MR cells are plated out in medium (Optimem, 2.5% FCS, 2 mM glutamine, 10 mM HEPES) in 96-well (or 384- or 1536-well) microliter plates the day before the assay, and are kept in a cell incubator (96% air humidity, 5% v/v CO₂, 37° C.). On the day of the assay, the substances to be tested are taken up in the aforementioned medium and added to the cells. About 10 to 30 minutes after addition of the test substances, the respective specific agonists of the steroid hormone receptors are added. After a further incubation time of 5 to 6 hours, the luciferase activity is measured with the aid of a video camera. The relative light units measured give a sigmoidal stimulation curve as a function of the substance concentration. The IC₅₀ values (in mol) are calculated with the aid of the computer program GraphPad PRISM (Version 3.02).

Compound of the formula (I): IC50:2.77e-008

M1a (S): IC50: 9.33e-006

M1b (R): IC50: >1.00e-005

### DESCRIPTION OF THE FIGURES

FIG. 1: Crystal structure of the compound of the formula M1b (R): (R)-4-(4-cyano-2-methoxyphenyl)-5-ethoxy-2,8-dimethyl-1,6-naphthyridine-3-carboxamide

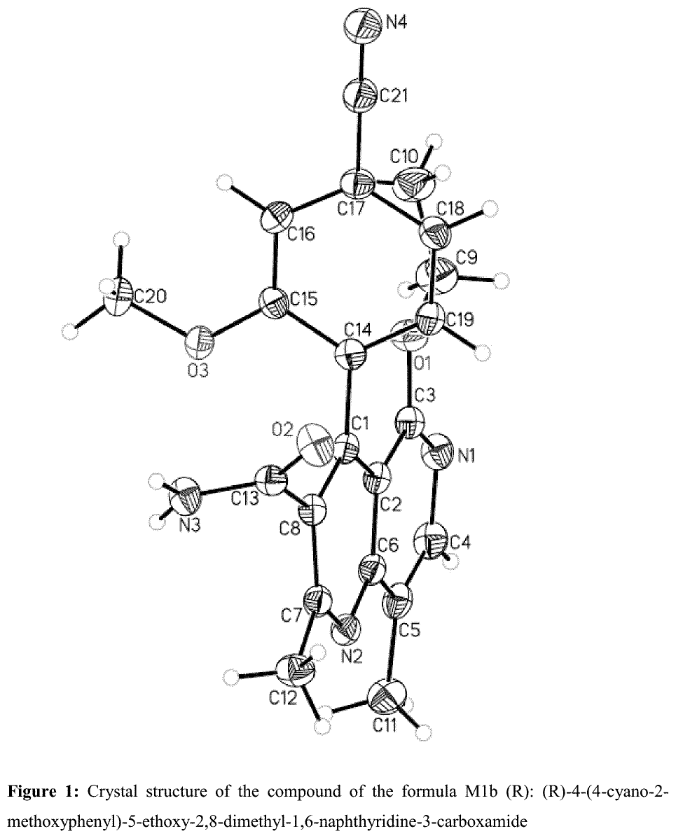

FIG. 2: Crystal structure of the compound of the formula M1b (R): (R)-4-(4-cyano-2-methoxyphenyl)-5-ethoxy-2,8-dimethyl-1,6-naphthyridine-3-carboxamide

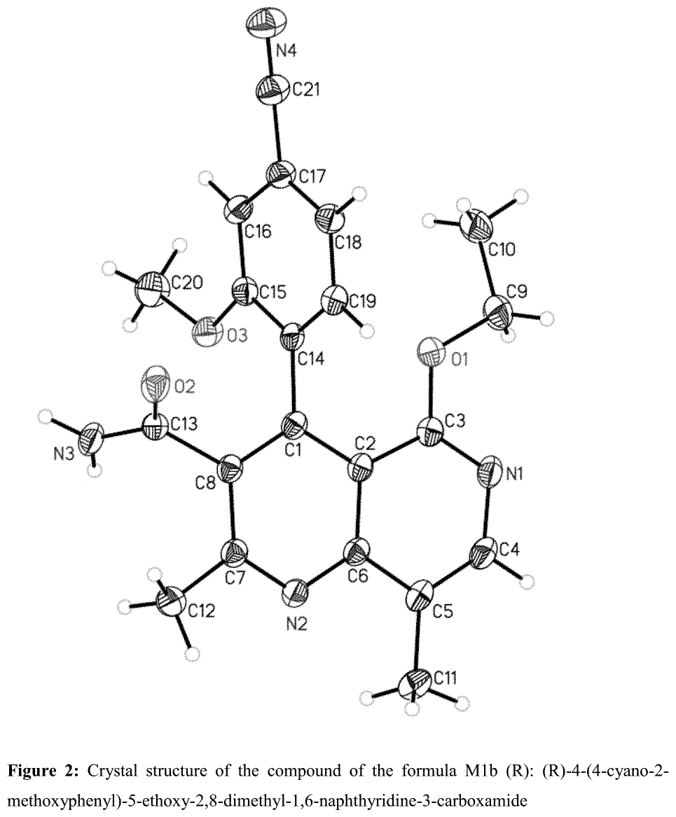

FIG. 3: CD spectrum of the compound of the formula M1b (R) (in acetonitrile)

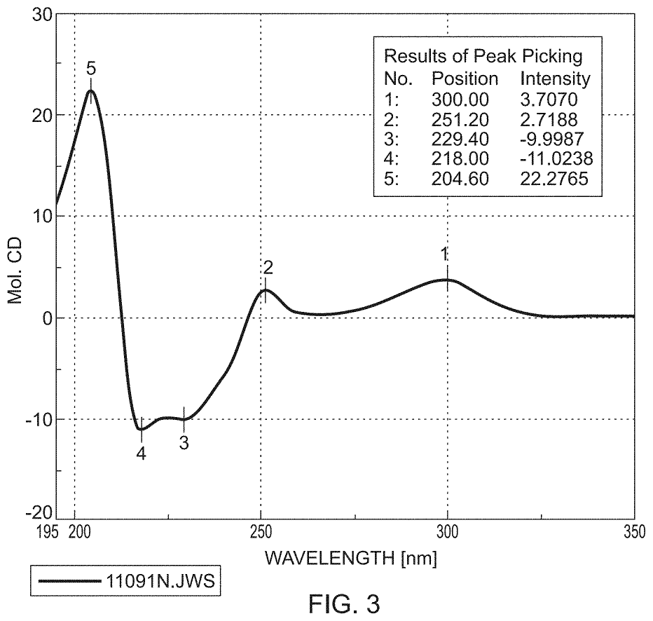

FIG. 4: CD spectrum of the compound of the formula M2b (R) (in acetonitrile)

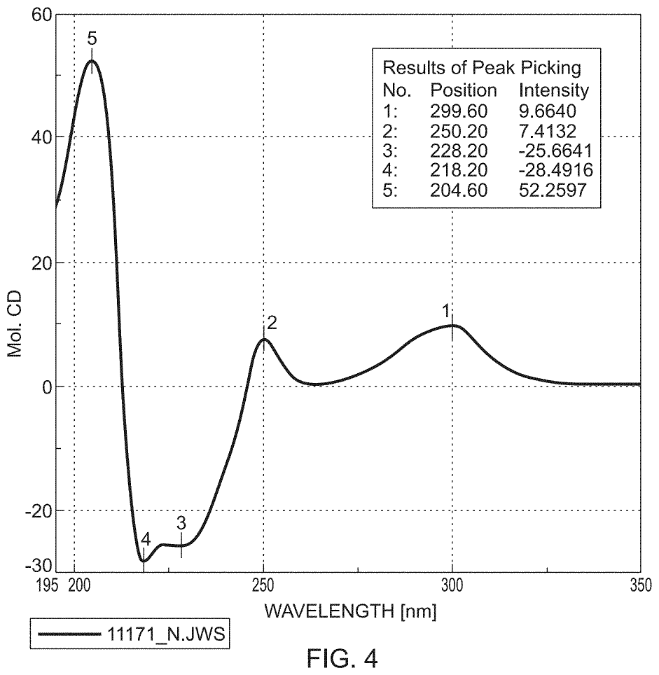

FIG. 5: CD spectrum of the compound of the formula M3b (R) (in acetonitrile)

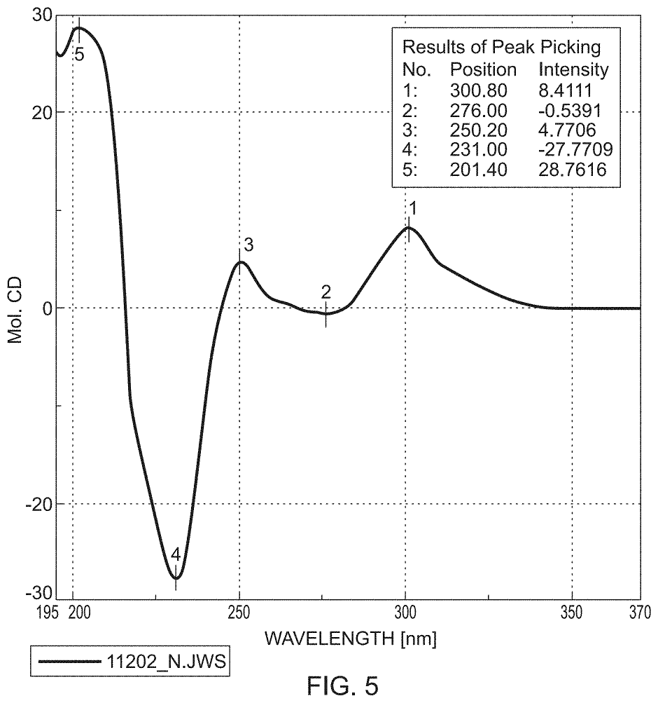

1. Compounds of the formulae

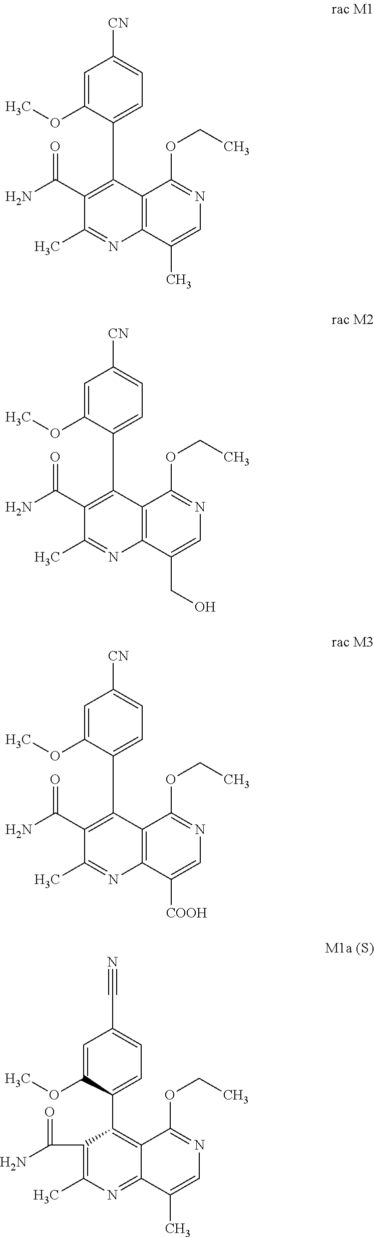

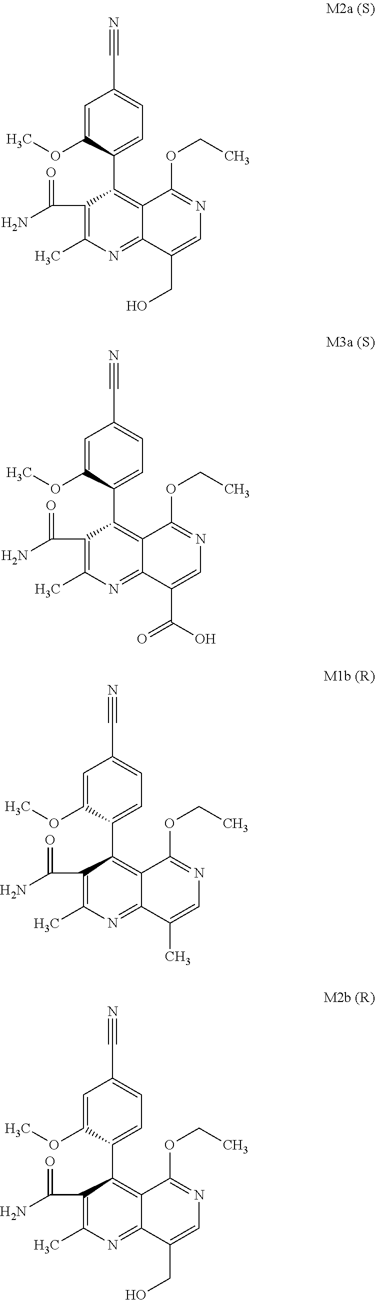

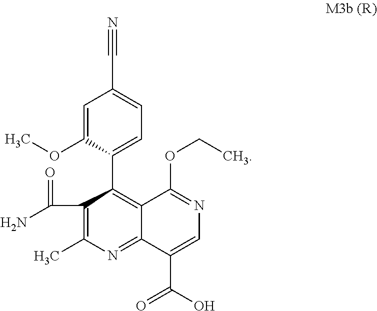

2. Method for preparing the compound of the formulae M1a (S) and M1b (R) 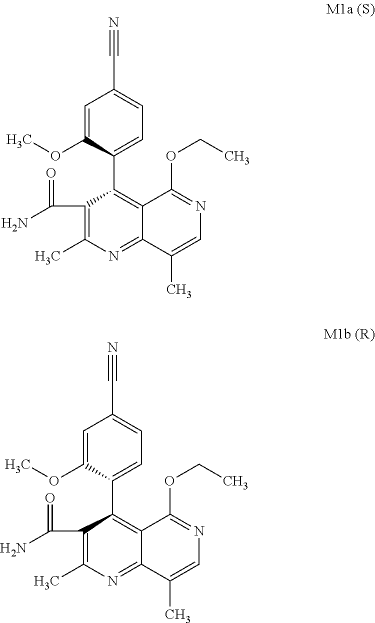
characterized in that the compound of the formula rac M1 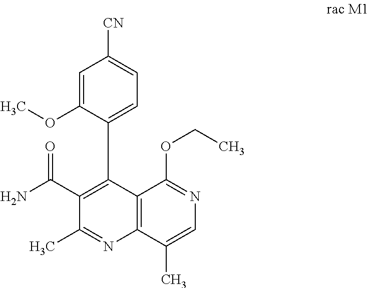 [SMILES: CCOc1ncc(C)c2nc(C)c(C(N)=O)c(-c3ccc(C#N)cc3OC)c12]
is prepared by oxidation of the compound of the formula rac (I) 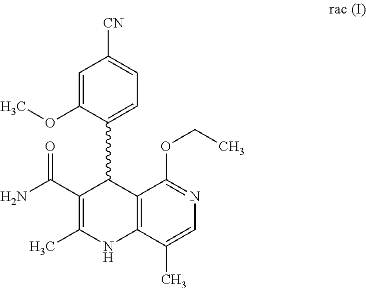 [SMILES: CCOc1ncc(C)c2c1C(c1ccc(C#N)cc1OC)C(C(N)=O)=C(C)N2]
and the racemate is separated into the enantiomers of the formulae M1a (S) and M1b (R) by chromatographic methods on a chiral phase.

3. Method for preparing the compound of the formulae M2a (S) and M2b (R) 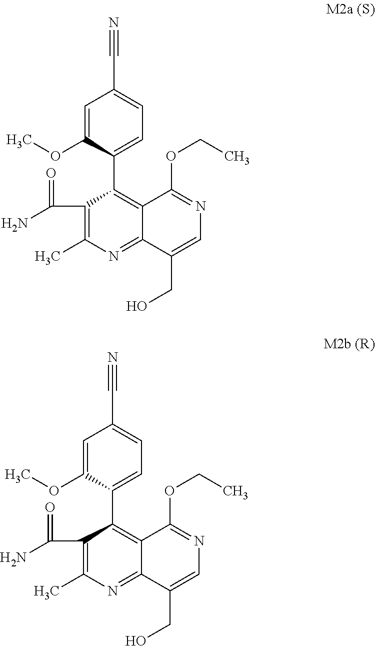
characterized in that the compound of the formula rac M2 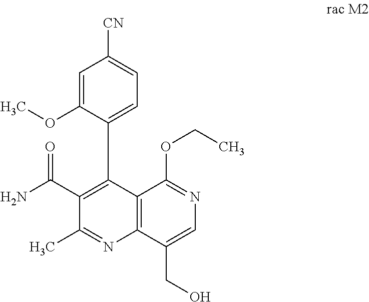 [SMILES: CCOc1ncc(CO)c2nc(C)c(C(N)=O)c(-c3ccc(C#N)cc3OC)c12]
is prepared by selective hydroxylation of the methyl group of the compound of the formula rac M1 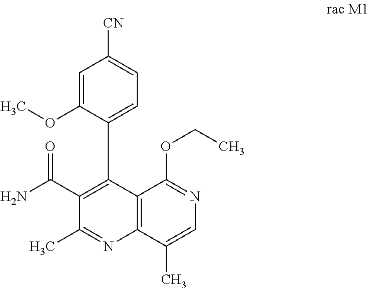 [SMILES: CCOc1ncc(C)c2nc(C)c(C(N)=O)c(-c3ccc(C#N)cc3OC)c12]
and the racemate is separated into the enantiomers of the formulae M2a (S) and M2b (R) by chromatographic methods on a chiral phase.

4. Method for preparing the compound of the formulae M3a (S) and M3b (R) 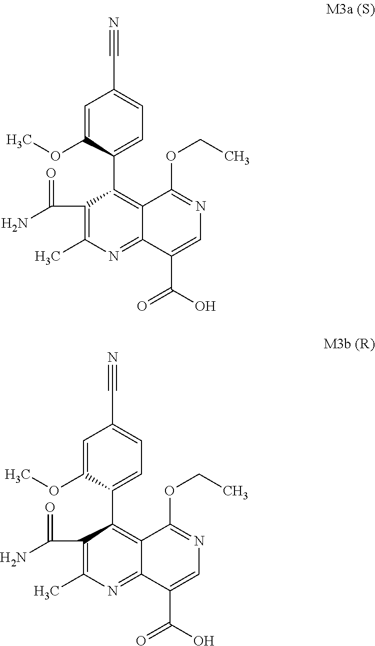
characterized in that the compound of the formula rac M3 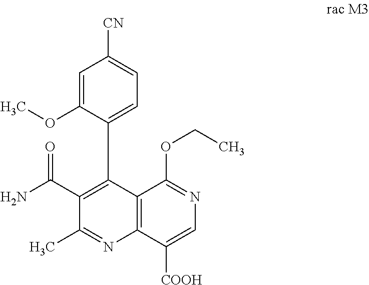 [SMILES: CCOc1ncc(C(=O)O)c2nc(C)c(C(N)=O)c(-c3ccc(C#N)cc3OC)c12]
is prepared by oxidation of the benzylic alcohol of the compound of the formula rac M2 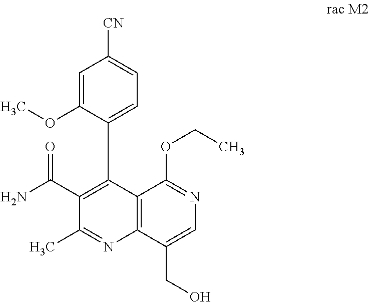 [SMILES: CCOc1ncc(CO)c2nc(C)c(C(N)=O)c(-c3ccc(C#N)cc3OC)c12]
and the racemate is separated into the enantiomers of the formulae M3a (S) and M3b (R) by chromatographic methods on a chiral phase.
